(19) United States
(12) Patent Application Publication
(10) Pub. No.: US 2001/0033836 A1
(43) Pub. Date: Oct. 25, 2001

(54) Transgenic autologous T-cell therapy in humans, and related compositions and kits

(76) Inventors: Geoff Symonds, Rose Bay (AU); Janet Macpherson, Leichhardt (AU)

Correspondence Address: AUDLEY A. CIAMPORCERO JR., JOHNSON & JOHNSON, ONE JOHNSON & JOHNSON PLAZA, NEW BRUNSWICK, NJ, 08933-7003, US

(21) Appl. No.: 09/815,237

(22) Filed: Mar. 22, 2001

Related U.S. Application Data
(63) Continuation of application No. 09/156,314, filed on Sep. 17, 1998.
(60) Provisional application No. 60/059,682, filed on Sep. 19, 1997.

(51) Int. Cl. A61K 48/00

(52) U.S. Cl. 424/93.21

(57) ABSTRACT

This invention provides a method of treating a subject afflicted with a disorder characterized by the presence of a unique epitopic locus in the subject, wherein there exists a therapeutic protein which is capable of ameliorating the effects of the disorder. This invention also provides a related pharmaceutical composition and kit.

Throughout this application, various publications are cited. The disclosure of these publications is hereby incorporated by reference into this application to describe more fully the state of the art to which this invention pertains.

### FIELD OF THE INVENTION

This invention relates to the use of transgenic autologous T-cells to treat disorders. The types of disorders treated are numerous and include, for example, autoimmune, allergic and other chronic inflammatory disorders, the most important of which being inflammatory disorders. Accordingly, the background section which follows is directed solely to inflammatory disorders.

### BACKGROUND OF THE INVENTION

Inflammatory Disorders Generally

Inflammatory disorders are a major health problem so debilitating that sufferers are unable to perform normal, everyday tasks. Currently available treatments include, for example, aspirin and other non-steroidal anti-inflammatory drugs, glucocorticoids like hydrocortisone, immune-modulating drugs such as cyclosporin A and even cytotoxic drugs including methotrexate and azothioprine. These treatments, however, are inadequate, with many patients experiencing significant side effects due to their non-specific action. These shortcomings place a large financial burden on the health care system and support networks, and are a major cause of lost productivity.

Diseases such as rheumatoid arthritis, where the therapeutic targets are multiple joints, have proven difficult to treat using traditional systemic delivery systems. The systemic: therapeutic protein levels required for sufficient protein to reach the joint often result in severe side effects (Jain and Lipsky, 1997). Intra-articular injections have been used to help overcome this problem, but the number of joints that can be treated simultaneously by this method is limited and repeat injections are required.

Numerous physiological systems play a role in the development and progression of inflammatory disorders. Such systems include cytokines and CD4⁺ T-cells (hereinafter “CD4⁺ cells”), to name two.

Cytokines

Cytokines are potent mediators of inflammation. They are required to maintain normal host defenses against infection. However, increased amounts of cytokines can lead to pathology such as tissue damage. Inflammation can be reduced in vivo by manipulation of the cytokine network. It is a general hypothesis, and there are certain specific examples showing, that alteration of the local concentration of a key cytokine will effect the inflammatory process. As a result, this local alteration of cytokine concentration has received consideration in the art as an approach to treat inflammatory disorders (Evans and Robbins, 1994; van den Berg, Joosten et al, 1994; Chernajovsky, Feldmann et al, 1995; Giladi, Raz et al, 1995; Kitamura, Burton et al, 1995; Kramer, Zhang et al, 1995; Raz, Dudler et al, 1995; Moritani, Yoshimoto et al, 1996; Chernajovsky, Adams et al, 1997; Mathisen, Yu et al, 1997; and Shaw, Lorens et al, 1997).

CD4⁺ Cells

CD4⁺ cells are vital components of the immune system. Each CD4⁺ cell has T-cell receptors which permit it to specifically recognize and bind to a particular epitope. Each CD4⁺ cell carries out surveillance as it circulates, “on the lookout” for the epitope to which it specifically binds. When the epitope is encountered, the CD4⁺ cell would release any secretable proteins, therapeutic or otherwise, for which its DNA encodes.

CD4⁺ cells may be further subdivided on the basis of antigen reactivity and expression of other cell surface markers such as adhesion molecules. The majority of mature CD4+ cells are immunologically naive not having encountered antigen before. Contact with specific antigen causes naive cells to proliferate rapidly and differentiate into a mixture of short-lived, activated, effector cells and long-lived memory cells. These cell populations may be defined immunophenotypically.

Activated, effector T-cells are CD3⁺CD4⁺CD45RO⁺CD25⁺HLA-DR⁺CD69⁺, memory cells are CD45RA^{lo} CD45RO^{hi}CD29⁺, while naive cells are CD45RA⁺ (Picker, Martin et al, 1994). Each CD4⁺ memory cell circulates throughout the lymphatics, migrating into non-lymphoid tissue (Rohnelt, Hoch et al, 1997) to carry out surveillance for the epitope to which the memory cell specifically binds. When the epitope is encountered, the memory cell becomes activated and proliferates locally. In addition, the CD4+ cell would also release any secretable proteins, therapeutic or otherwise, for which its DNA encodes. While this surveillance occurs in a constitutive manner, the presence of certain cell surface adhesion molecules and integrins enhances the migration to particular tissues including skin, gut and lung (Picker, Martin et al, 1994; Babi, Soler et al, 1995; and Meenan, Spaans et al, 1997), and synovium (Lazarovits and Karsh, 1993; Yokota, Murata et al, 1995).

In normal peripheral blood, the ratio of CD4⁺:CD8⁺ cells is approximately 2:1 and it is straightforward to isolate a population of cells enriched for CD4⁺ cells. Mononuclear cells are isolated by density gradient centrifugation using Ficoll-Hypaque (Coligan, Kruisbeek et al, 1997). Monocytes may be removed by adherence, and the remaining lymphocyte population can be enriched for CD4⁺ cells by either positive or negative selection methods.

A variety of techniques and devices based on monoclonal antibodies are available. For positive selection of CD4⁺ cells, the lymphocytes are reacted with a monoclonal CD4 antibody and the CD4-reactive cells are retained. For negative selection, the lymphocytes are reacted with monoclonal antibodies against other cell surface markers such as CD8 and CD19 and the non-reactive cells are retained. Selection may occur in liquid phase, using a fluorescent-tagged antibody and fluorescent-activated cell sorting (FACS) of reactive and non-reactive cells, or complement-mediated lysis of reactive cells. Alternatively, the antibody may be immobilized on a solid-phase such as magnetic particles, polystyrene flask, or other material packed into a column (Coligan, Kruisbeek et al, 1997) and such systems are commercially available. Both the reactive (positive selection) and non-reactive (negative selection) populations may be recovered.

CD4⁺ cells have been further subdivided on a functional basis into Th1 and Th2 cells. This classification was based on the differential production of various cytokines by T-cell clones, and more recently by single cells (Bucy, Panoskaltsis-Mortari et al, 1994, Vikingsson, Pederson et al, 1994). Th1 cells produce interleukin-2 and interferon-γ while Th2 cells produce interleukin-4 and interleukin-5. A deficiency of Th2 cells has been implicated in the pathogenesis of autoimmune diseases while Th2 cells are over-represented in allergic conditions (Romagnani, 1994).

CD4⁺ cells are also long-lived with evidence to indicate their survival for months or even years (Picker and Butcher, 1992; Tough and Sprent, 1995). The continued production of therapeutic protein by these cells over a long period would reduce the number of treatment cycles required.

CD4⁺ cells have already been used for gene therapy of abnormalities such as adenosine deaminase deficiency (Blaese, Culver et al, 1995; Mullen, Snitzer et al, 1996), and therapy for human immunodeficiency virus infection (Walker, Blaese et al, 1993). In these instances, the total CD4⁺ T-cell population was used, without first having to enrich this population for CD4⁺ cells specific for a particular epitope.

Animal Models for Human Disease

Limited success has been shown using transgenic CD4⁺ cells to treat inflammatory disorders in animal models. Specifically, transgenic CD4⁺ cells have been used in rat to delay onset of rat experimental autoimmune encephalomyelitis (EAE) model (corresponding to human multiple sclerosis) and rat experimental autoimmune neuritis (EAN) model (corresponding to human Guillain-Barre syndrome) (Refs. 32 and 15, respectively). Transgenic CD4⁺ cells have also been used in mouse both to delay onset and treat mouse EAE model (corresponding to human multiple sclerosis), delay onset of diabetes in NOD mouse (corresponding to human insulin-dependent diabetes mellitus), treat arthritis in DBA/1 mouse collagen-induced arthritis (CIA) (corresponding to human rheumatoid arthritis), and delay onset of athritis in SCID mouse CIA (corresponding to human rheumatoid arthritis) (Refs. 17, 21, 6 and 6, respectively).

However, no human studies have been performed showing therapeutic success on a disease using CD4⁺ cells enriched for cells specific for a particular epitopic locus characteristic of the disease. The animal studies to date are not predictive of success in humans for the following reasons. First, animal and human cytokine systems differ from one another. Specifically, human Th1 and Th2 cells both produce IL-10, whereas in mouse, for example, only Th2 cells produce IL-10. This distinction is significant being that Th1 cells play an important role in human inflammation. Second, animal and human T-cell subsets differ, as defined by their surface markers. For these reasons animal models, and mouse models in particular, are not of predictive value for showing success in humans using enriched CD4⁺ cell therapy.

### SUMMARY OF THE INVENTION

This invention provides a method of treating a human subject afflicted with a disorder characterized by the presence of a unique epitopic locus in the subject, wherein there exists a therapeutic protein capable of ameliorating the effects of the disorder, the method comprising the steps of (a) isolating CD4⁺ cells from the subject; (b) treating the isolated CD4⁺ cells so as to enrich the population of cells therein which specifically bind to the unique epitopic locus; (c) forming transgenic CD4⁺ cells by introducing into the treated CD4⁺ cells a nucleic acid molecule encoding the therapeutic protein, wherein the nucleic acid molecule stably propagates to progeny transgenic CD4⁺ cells and causes the expression and extracellular placement of the therapeutic protein; and (d) administering to the subject a therapeutically effective dose of the resulting transgenic CD4⁺ cells.

This invention also provides a pharmaceutical composition for treating a human subject afflicted with a disorder characterized by the presence of a unique epitopic locus in the subject, wherein there exists a therapeutic protein capable of ameliorating the effects of the disorder, the composition comprising (a) CD4⁺ cells derived from the subject which specifically bind to the unique epitopic locus, and which have introduced thereinto a nucleic acid molecule encoding the therapeutic protein, wherein the nucleic acid molecule is stably transmitted to progeny CD4⁺ cells and causes the expression and extracellular placement of the therapeutic protein; and (b) a pharmaceutically acceptable carrier.

This invention further provides a method of treating a human subject afflicted with a disorder characterized by the presence of a unique epitopic locus in the subject, wherein there exists a therapeutic protein capable of ameliorating the effects of the disorder, the method comprising the step of administering to the subject a therapeutically effective dose of the instant pharmaceutical composition.

Finally, this invention provides a kit for use in practicing the instant method of treatment comprising (a) a suitable tissue culture medium for growing CD4⁺ cells, and (b) a suitable factor for inducing CD4⁺ cell growth.

### BRIEF DESCRIPTION OF THE FIGURE

FIG. 1 shows the expression of therapeutic genes in human T-cell lines. Jurkat cells transfected with genes encoding anti-inflamatory cytokines inhibit the production of the pro-inflammatory cytokines TNFα and IL-6 by a murine macrophage cell line. Jurkat represents the parental cell line, while J-L9XL are Jurkat cells transfected with the vector control, J-TGFS and J-TGFD are cells transfected with two TGFβ1 constructs, and J-LmIL4, J-hIL10, J-IL1ra and J-crmB are cells that were transfected with murine IL-4, human IL-10, human IL-1 receptor antagonist and CPV crmB contructs respectively. All cells except “no LPS”, were stimulated with 2 μg/mL LPS.

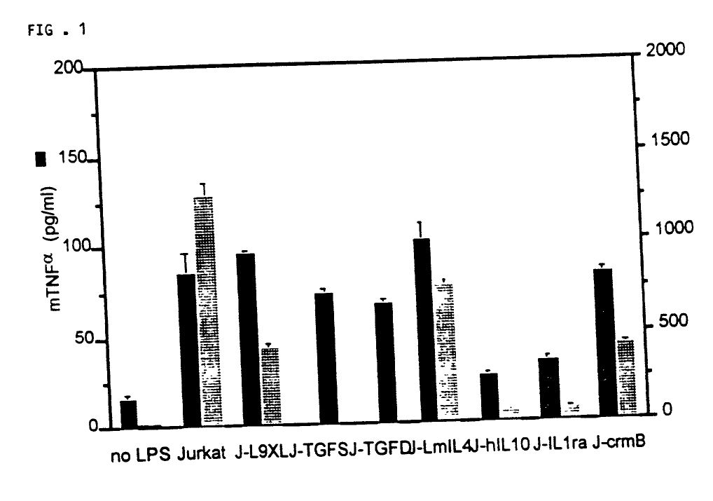

### DETAILED DESCRIPTION OF THE INVENTION

This invention relates to the use of transgenic autologous CD4⁺ cells to treat certain disorders in humans, chiefly inflammatory disorders. This invention is characterized, in part, by several unique features. First, the CD4⁺ cells used are taken from the human subject being treated. Second, the population of CD4⁺ cells taken from the subject are enriched for cells which specifically recognize a unique epitopic region characteristic of the disorder being treated. Finally, the enriched CD4⁺ cells are recombinantly engineered to express a therapeutic protein known to ameliorate the disorder.

The advantages of the instant invention are several-fold. It permits the site-specific delivery of a therapeutic protein via a more specific and effective procedure. Also, the delivery of the therapeutic protein is continuous over a long period of time, and allows a high concentration of the protein to be delivered to the site of the disorder. This high concentration would otherwise be difficult and dangerous (e.g. resulting in side-effects) to achieve by known methods of treating humans. Finally, the instant method carries with it a relatively low treatment cost over time, since it overcomes the need for repeated treatments, the resulting costs due to doctor time and hospital time, and the costs due to the subject's own loss of productivity.

Specifically, this invention provides a method of treating a human subject afflicted with a disorder, wherein the disorder is characterized by the presence of a unique epitopic locus in the subject, and there exists a therapeutic protein capable of ameliorating the effects of the disorder, the method comprising the steps of

(a) isolating CD4⁺ cells from the subject;

(b) treating the isolated CD4⁺ cells so as to enrich the population of cells therein which specifically bind to the unique epitopic locus;

(c) forming transgenic CD4⁺ cells by introducing into the treated CD4⁺ cells a nucleic acid molecule encoding the therapeutic protein, wherein the nucleic acid molecule is stably transmitted to progeny CD4⁺ cells and causes the expression and extracellular placement of the therapeutic protein; and

(d) administering to the subject a therapeutically effective dose of the resulting transgenic CD4⁺ cells.

The disorder treated by the instant method can be any human disorder characterized by the presence of a unique epitopic locus in the subject, and for which there exists a therapeutic protein capable of ameliorating the effects of the disorder. In the preferred embodiment, the disorder is an inflammatory disorder.

In one embodiment, the inflammatory disorder is allergic inflammation or an autoimmune disorder. Autoimmune disorders include, but are not limited to, rheumatoid arthritis, insulin-dependent diabetes mellitus, multiple sclerosis, myasthenia gravis, Crohn's disease, autoimmune nephritis, primary biliary cirrhosis and psoriasis. In the preferred embodiment, the autoimmune disorder is rheumatoid arthritis. In one embodiment, the inflammatory disorder is Crohn's disease.

As used herein, a “unique epitopic locus” means the surface area on a single antigenic molecule, or formed by a plurality of antigenic molecules, which (a) exists at or near the location of the disorder in the afflicted subject, (b) does not exist at or near this site in an unafflicted subject, and (c) can be recognized by and specifically bound to CD4⁺ cells present in the afflicted subject. In the preferred embodiment, the unique epitopic locus exists only at or near the location of the disorder in the afflicted subject, and not at any other location. For example, a unique epitopic locus can be the surface area of exposed and degraded collagen type II at and near the inflamed joint of a subject afflicted with arthritis. By “near” the location of the disorder, it is meant a distance close enough to the location of the disorder for a therapeutically effective amount of the therapeutic protein to be delivered to the location of the disorder.

Examples of disorders and their corresponding unique epitopic loci (or material containing or forming same) include the following: rheumatoid arthritis (collagen type II, synovial material); multiple sclerosis (myelin basic protein, proteolipid protein, myelin oligodendrocyte glycoprotein); myasthenia gravis (nicotinic acetylcholine receptor); insulin-dependent diabetes mellitus (proinsulin, glutamic acid decarboxylase); primary billiary cirrhosis (nuclear pore complex proteins, 2-oxo-acid dehydrogenase); psoriasis (cutaneous leukocyte antigen, Streptococcal M protein); Crohn's disease (α4β7 integrin); Guillain-Barre syndrome (peripheral myelin protein); autoimmune gastritis (gastric H⁺/K⁺ ATPase, intrinsic factor); Addison's disease (21-hydroxylase); Grave's disease (thyroid stimulating hormone receptor); Hashimoto's thyroiditis (thyroid peroxidase); and autoimmune uveitis (S-antigen).

The therapeutic protein used in the instant method can be any protein capable of ameliorating the effects of a disorder characterized by the presence of a unique epitopic locus in the subject. Therapeutic proteins, together with examples of the treated disorders, include, but are not limited to, TGF_{β1} (rheumatoid arthritis, multiple sclerosis, myathenia gravis, glomerulonephritis, colitis, uveitis), interleukin-4 (rheumatoid arthritis, multiple sclerosis), interleukin-10 (rheumatoid arthritis, diabetes), interleukin-13 (rheumatoid arthritis), interleukin-1 receptor agonist (rheumatoid arthritis), soluble interleukin 1 receptor (graft versus host disease), soluble tumor necrosis factor alpha receptor (rheumatoid arthritis, multiple sclerosis), and cow pox virus crmB (rheumatoid arthritis). In the preferred embodiment, the therapeutic protein is IL-10.

As used herein, “ameliorating” the effects of the disorder means (a) stopping, reversing or reducing the progression of the disorder, and/or (b) stopping, reversing or reducing the progression of symptoms of the disorder.

Methods of isolating CD4⁺ cells from humans are well known in the art (Coligan, Kruisbeek et al, 1997). Methods of treating isolated CD4⁺ cells so as to enrich the population of cells therein which specifically bind to a unique epitopic locus are also well known in the art (Pawelec, 1993).

As used herein, a population of CD4⁺ cells is “enriched” for CD4⁺ cells which specifically bind to a unique epitopic locus if the percentage of cells which specifically bind to the unique epitopic locus after treatment of the cells (%A) is at least about 2-fold greater than the percentage of CD4⁺ cells which specifically bind to the unique epitopic locus before treatment of the cells (%B). In other words, in such an enriched population of CD4⁺ cells, the ratio of %A to %B is at least about 2. In the preferred embodiment, the ratio of %A to %B is at least about 10.

Methods of forming transgenic cells, including CD4⁺ cells, by introducing nucleic acid molecules thereinto are well known to those of skill in the art. Such methods include, for example, the use of viral vectors and calcium phosphate co-precipitation (Miller, Miller et al, 1993; Finer, Dull et al, 1994; Imbert, Costello et al, 1994; Mavilio, Ferrari et al, 1994; Nagoya, Greenberg et al, 1994; Sun, Pyati et al, 1995; Asami, Germeraad et al, 1996; Mullen, Snitzer et al, 1996; Rudoll, Phillips et al, 1996; Sharma, Cantwell et al, 1996; and Behr, 1994).

The nucleic acid molecule encoding the therapeutic protein can be DNA or RNA. In the preferred embodiment, the nucleic acid molecule is a recombinant nucleic acid molecule. As used herein, a recombinant nucleic acid molecule is a nucleic acid molecule which does not occur as an individual molecule in nature and which is obtained through the use of recombinant technology. Examples of recombinant nucleic acid molecules include, for example, expression vectors and plasmids.

Numerous nucleic acid vectors for expressing the instant therapeutic proteins may be employed. Such vectors, including pLNL6 (a retroviral vector), are well known in the art. For example, one class of vectors utilizes DNA elements which are derived from or based on animal viruses such as bovine papilloma virus, polyoma virus, adenovirus, vaccinia virus, baculovirus, retroviruses (RSV, MMTV or MoMLV), Semliki Forest virus or SV40 virus. Methods for using markers (such as resistance to a certain toxin) which facilitate the selection of transfected or infected host cells are also well known. The selectable marker gene can be either directly linked to the DNA sequences to be expressed, or introduced into the same cell by cotransformation.

Expression vectors require regulatory elements for expression. These elements include, for example, promoter sequences to cause binding of RNA polymerase and translation initiation sequences for ribosome binding. Additional elements may also be needed for optimal synthesis of mRNA. These additional elements may include splice signals, as well as enhancers and termination signals. For example, eukaryotic expression vectors can include a heterologous or homologous promoter for RNA polymerase II, a downstream polyadenylation signal, the start codon AUG, and a termination codon for detachment of the ribosome. Such expression vectors may be obtained commercially or assembled from the sequences described by methods well known in the art.

In the instant invention, the therapeutic protein which is placed extracellularly can be secreted from the CD4⁺ cell in soluble form, or alternatively, exist as a membrane-bound protein on the surface of the CD4⁺ cell.

As used herein, administering may be effected or performed using any of the various methods known to those skilled in the art. The administering may comprise administering intravenously, intramuscularly, and subcutaneously. In the preferred embodiment, the administering is performed intravenously.

Methods of determining therapeutically effective doses for administering cell-based treatments in humans are known in the art. The effective dose for administering a cell-based therapeutic would be determined mathematically from the results of animal studies. In one embodiment, the effective dose is from about 10⁵ to about 10¹⁰ cells for a 75 kg adult. In another embodiment, the therapeutically effective dose of transgenic CD4⁺ cells is between from about 10⁶ to about 10⁹ cells for a 75 kg adult.

This invention also provides a pharmaceutical composition for treating a human subject afflicted with a disorder characterized by the presence of a unique epitopic locus in the subject, wherein there exists a therapeutic protein capable of ameliorating the effects of the disorder, the composition comprising

(a) CD4⁺ cells derived from the subject which specifically bind to the unique epitopic locus, and which have introduced thereinto a nucleic acid molecule encoding the therapeutic protein, wherein the nucleic acid molecule is stably transmitted to progeny CD4⁺ cells and causes the expression and extracellular placement of the therapeutic protein; and

(b) a pharmaceutically acceptable carrier.

Pharmaceutically acceptable carriers are well known to those skilled in the art and include, but are not limited to, 0.01-0.1M and preferably 0.05M phosphate buffer or 0.8% saline. Additionally, such pharmaceutically acceptable carriers may be aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's or fixed oils. Intravenous vehicles include fluid and nutrient replenishers, electrolyte replenishers such as those based on Ringer's dextrose, and the like. Preservatives and other additives may also be present, such as, for example, antimicrobials, antioxidants, chelating agents, inert gases, albumin and the like.

This invention also provides a method of treating a human subject afflicted with a disorder characterized by the presence of a unique epitopic locus in the subject, wherein there exists a therapeutic protein capable of ameliorating the effects of the disorder, the method comprising the step of administering to the subject a therapeutically effective dose of the instant pharmaceutical composition.

Finally, this invention provides a kit for use in practicing the instant method of treatment comprising (a) a suitable tissue culture medium for growing CD4⁺ cells, and (b) a suitable factor for inducing CD4⁺ cell growth.

The tissue culture medium used can be obtained commercially, and can be frozen or lyophilized. Factors for inducing CD4⁺ cell growth are known in the art, and include by way of example, IL-2, either frozen or lyophilized.

In another embodiment, the instant kit further comprises one or more of the following: (a) T-cell isolation materials including, but not limited to, fluid collection tubes and bags, liquid density gradient medium, and a T-cell selection device; (b) gene introduction materials including, but not limited to, an expression vector encoding for a therapeutic protein, and a helper protein such as aqueous protamine sulfate; (c) a cell cuture growth expansion device; (d) additional cell culture materials including, but not limited to, stimulatory molecules such as aqueous OKT3-anti-CD3 antibody known in the art, and antigen for which the CD4⁺ cells isolated are to be specific (lyophilized) or a means to prepare same; and (e) an infusion bag.

The components of the instant kit can be in the same or separate compartments. In the preferred embodiment, the kit further comprises instructions for use.

This invention will be better understood by reference to the Experimental Details which follow, but those skilled in the art will readily appreciate that the specific experiments detailed are only illustrative of the invention as described more fully in the claims which follow thereafter.

### EXPERIMENTAL DETAILS

### Example 1

A. Treating Rheumatoid Arthritis in Humans with TGF_{β1}

Peripheral blood is collected from an individual with rheumatoid arthritis, and the CD4⁺ cells isolated. These cells are cultured with chicken or bovine collagen type II or autologous synovial fluid in the presence of autologous Epstein Barr virus-transformed B-cells (LCL), or dendritic cells or other antigen-presenting cells. CD4⁺ cells reactive to collagen type II or antigens within synovial fluid are then expanded in the presence of interleukin-2. The gene encoding an anti-inflammatory cytokine molecule, e.g. IL-10, is introduced into the CD4⁺ cells via a retroviral vector. The transduced cells are expanded and injected into patients via the intravenous route.

Cells with T-cell receptor specificity for collagen type II or other joint or synovial proteins would be expected to circulate and migrate into multiple joints where, due to joint degradation and cartilage damage, collagen type II has been exposed. These cells would remain within the joint, proliferate and produce recombinant IL-10 protein which is released into the joint and acts locally to reduce the release of inflammatory cytokines by others cells within the local environment, in particular, synoviocyte and macrophage release of TNFα, IL-1β and other inflammatory and joint-degrading matrix metaloproteinases. Cells that exit the joint and travel to lymph nodes are then available for ongoing tissue surveillance, reactivation and proliferation at the site of joint damage when there is a “flare up” of disease.

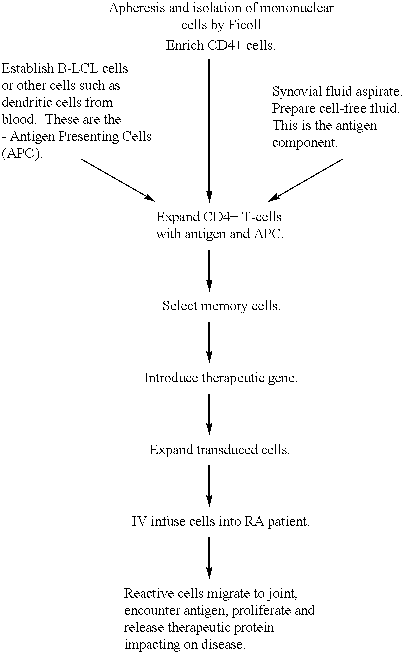

### Example 2

A. Murine Arthritis Model

Although not predictive of success in humans, the murine model of collagen-induced arthritis has advantages as a model of human rheumatoid arthritis; disease does not occur in all animals and there is chronic polyarthritis. CD4⁺ cells are manipulated in vitro in the presence of autologous antigen-presenting cells and collagen type II, to induce proliferation of cells with known CD4+ cell receptor specificity. The collagen-reactive CD4⁺ cells are engineered to express an anti-inflammatory protein, expanded, and reinjected into mice with collagen-induced arthritis.

It is possible to introduce into CD4⁺ cells gene constructs encoding anti-inflammatory molecules including, but not limited, to TGF_{β1,} interleukin-10 (IL-10), interleukin-4 (IL-4), interleukin-1 receptor antagonist (IL-1ra), and the viral crmB gene, so that they are expressed. Also, in tissue culture, CD4⁺ cells expressing IL-10 or IL-1ra inhibited the release of TNF_{α} and IL-6 by lipopolysaccharide-stimulated macrophage cells. The CD4⁺ cells with T-cell receptor specificity for collagen type II are expected to migrate to the inflamed joints upon infusion into a diseased animal. Within the joint, the production of an anti-inflammatory molecule is expected to inhibit macrophage activation and release of TNF_{α}, IL-1β and other inflammatory and destructive molecules, thus reducing inflammation and joint destruction. The therapeutic effect is assessed by physical examination of swollen joints, the number of involved joints, and then confirmed by histological examination.

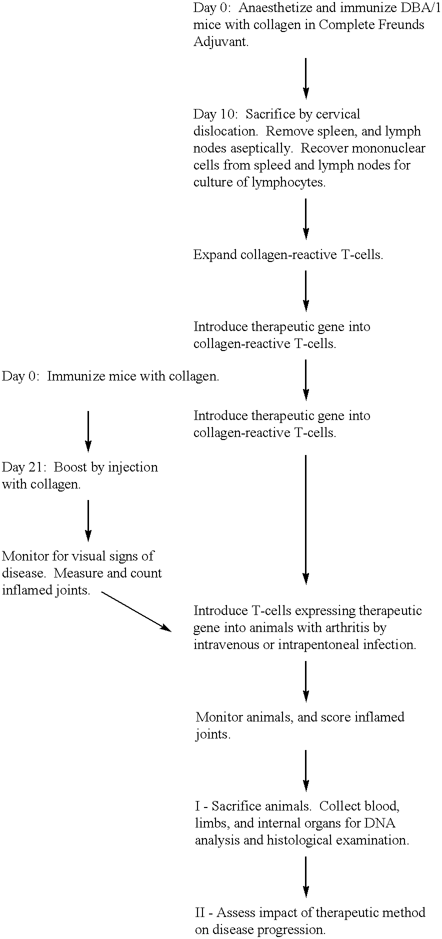

### REFERENCES

1. Asami, N., W. T. V. Germeraad, S. Fujimoto, S. Nagai, T. Izumi, Y. Katsura, Gene transduction into murine primitive hematopoietic cells with 2-gene retroviral vectors using a transwell coculture system. European Journal of Haematology, 1996. 57(4): 278-285.

2. Babi, L. F. S., M. T. P. Soler, C. Hauser, K. Blaser, Skin-homing T cells in human cutaneous allergic inflammation. Immunologic Research, 1995. 14(4): 317-324.

3. Behr, J. P., Gene transfer with synthetic cationic amphiphiles: prospects for gene therapy. Bioconjugate Chem., 1994. 5(5): 382-389.

4. Blaese, R. M., K. W. Culver, A. D. Miller, C. S. Carter, T. Fleisher, M. Clerici, G. Shearer, L. Chang, Y. Chiang, P. Tolstoshev, J. J. Greenblatt, S. A. Rosenberg, H. Klein, M. Berger, C. A. Mullen, W. J. Ramsey, L. Muul, R. A. Morgan, W. F. Anderson, T lymphocyte-directed gene therapy for ADA⁻ SCID: Initial trial results after 4 years. Science, 1995. 270: 475-480.

5. Bucy, R. P., A. Panoskaltsis-Mortari, G. Q. Huang, J. M. Li, L. Karr, M. Ross, J. H. Russell, K. M. Murphy, C. T. Weaver, Heterogeneity of single cell cytokine gene expression in clonal T cell populations. J. Exp. Med., 1994. 180(4): 1251-1262.

6. Chernajovsky, Y., G. Adams, K. Triantaphyllopoulos, M. F. Ledda, O. L. Podhajcer, Pathogenic lymphoid cells engineered to express TGFβ1 ameliorate disease in a collagen-induced arthritis model. Gene Ther, 1997. 4(6): 553-559.

7. Chernajovsky, Y., M. Feldmann, R. N. Maini, Gene therapy of rheumatoid arthritis via cytokine regulation: future perspectives. Br. Med. Bull., 1995. 51(2): 503-516.

8. Coligan, J. E., A. M. Kruisbeek, D. H. Margulies, E. M. Shevach, W. Strober, eds. Current Protocols In Immunology. 1997, John Wiley & Sons, Inc.

9. Evans, C. H., P. D. Robbins, Gene therapy for arthritis, in Gene Therapeutics: Methods and applications of direct gene transfer, J. A. Wolff, Editor. 1994, Birkhäuser Boston: Cambridge, Mass. p. 320-343.

10. Finer, M. H., T. J. Dull, L. Qin, D. Farson, M. R. Roberts, kat: a high-efficiency retroviral transduction system for primary human T lymphocytes. Blood, 1994. 83(1): 43-50.

11. Giladi, E., E. Raz, F. Karmeli, E. Okon, D. Rachmilewitz, Transforming growth factor-beta gene therapy ameliorates experimental colitis in rats. Eur J Gastroenterol Hepatol, 1995. 7(4): 341-347.

12. Imbert, A. M., R. Costello, J. Imbert, P. Mannoni, C. Bagnis, Highly efficient retroviral gene transfer into human primary T lymphocytes derived from peripheral blood. Cancer Gene Ther, 1994. 1(4): 259-265.

13. Jain, R., P. E. Lipsky, Treatment of rheumatoid arthritis. Med Clin North Am, 1997. 81(1): 57-84.

14. Kitamura, M., S. Burton, J. English, H. Kawachi, L. G. Fine, Transfer of a mutated gene encoding active transforming growth factor-β1 suppresses mitogenesis and IL-1 response in the glomerulus. Kidney Int., 1995. 48(6): 1747-1757.

15. Kramer, R., Y. P. Zhang, J. Gehrmann, R. Gold, H. Thoenen, H. Wekerle, Gene transfer through the blood-nerve barrier: NGF-engineered neuritogenic T lymphocytes attenuate experimental autoimmune neuritis. Nature Medicine, 1995. 1(11): 1162-1166.

16. Lazarovits, A. I., J. Karsh, Differential expression in rheumatoid synovium and synovial fluid of α4 β7 integrin. A novel receptor for fibronectin and vascular cell adhesion molecule-1. J. Immunol., 1993. 151(11): 6482-6489.

17. Mathisen, P. M., M. Yu, J. M. Johnson, J. A. Drazba, V. K. Tuohy, Treatment of experimental autoimmune encephalomyelitis with genetically modified memory T cells. J Exp Med, 1997. 186(1): 159-164.

18. Mavilio, F., G. Ferrari, S. Rossini, N. Nobili, C. Bonini, G. Casorati, C. Traversari, C. Bordignon, Peripheral blood lymphocytes as target cells of retroviral vector-mediated gene transfer. Blood, 1994. 83(7): 1988-1997.

19. Meenan, J., J. Spaans, T. A. Grool, S. T. Pals, G. N. J. Tytgat, S. J. H. Vandeventer, Altered expression of alpha(4)beta(7), a gut homing integrin, by circulating and mucosal t cells in colonic mucosal inflammation. Gut, 1997. 40(2): 241-246.

20. Miller, A. D., D. G. Miller, J. V. Garcia, C. M. Lynch, Use of retroviral vectors for gene transfer and expression, in Recombinant DNA, Pt H, R. Wu, Editor. 1993, Academic Press Inc., p. 581-599.

21. Moritani, M., K. Yoshimoto, S. Ii, M. Kondo, H. Iwahana, T. Yamaoka, T. Sano, N. Nakano, H. Kikutani, M. Itakura, Prevention of adoptively transferred diabetes in nonobese diabetic mice with IL-10-transduced islet-specific Th1 lymphocytes: a gene therapy model for autoimmune diabetes. Journal of Clinical Investigation, 1996. 98(8): 1851-1859.

22. Mullen, C. A., K. Snitzer, K. W. Culver, R. A. Morgan, W. F. Anderson, R. M. Blaese, Molecular analysis of T lymphocyte-directed gene therapy for adenosine deaminase deficiency: long-term expression in vivo of genes introduced with a retroviral vector. Hum Gene Ther, 1996. 7(9): 1123-1129.

23. Nagoya, S., P. D. Greenberg, C. Yee, K. E. Weisser, H. Sugawara, M. B. Widmer, J. Slack, S. K. Dower, S. D. Lupton, R. W. Overell, Helper T cell-independent proliferation of CD8⁺ cytotoxic T lymphocytes transduced with an IL-1 receptor retrovirus. J. Immunol., 1994. 153(4): 1527-1535.

24. Pawelec, G., Cloning and propagation of human T lymphocytes, in Tumour Immunobiology. A practical approach, G. Gallagher, R. C. Rees, and C. W. Reynolds, Editors. 1993, IRL Press: Oxford. p. 131-141.

25. Picker, L. J., E. C. Butcher, Physiological and molecular mechanisms of lymphocyte homing. Annu Rev Immunol, 1992. 10: 561-591.

26. Picker, L. J., R. J. Martin, A. Trumble, L. S. Newman, P. A. Collins, P. R. Bergstresser, D. Y. M. Leung, Differential expression of lymphocyte homing receptors by human memory effector T cells in pulmonary versus cutaneous immune effector sites. European Journal of Immunology, 1994. 24(6): 1269-1277.

27. Raz, E., J. Dudler, M. Lotz, S. M. Baird, C. C. Berry, R. A. Eisenberg, D. A. Carson, Modulation of disease activity in murine systemic lupus erythematosus by cytokine gene delivery. Lupus, 1995. 4(4): 286-292.

28. Rohnelt, R. K, G. Hoch, Y. Reiss, B. Engelhardt, Immunosurveillance modelled in vitro: Naive and memory T cells spontaneously migrate across unstimulated microvascular endothelium. Int Immunol, 1997. 9(3): 435-450.

29. Romagnani, S., Lymphokine production by human T cells in disease states. Annu. Rev. Immunol., 1994. 12: 227-257.

30. Rudoll, T., K. Phillips, S. W. Lee, S. Hull, O. Gaspar, N. Sucgang, E. Gilboa, C. Smith, High-efficiency retroviral vector mediated gene transfer into human peripheral blood CD4⁺ T lymphocytes. Gene Ther, 1996. 3(8): 695-705.

31. Sharma, S., M. Cantwell, T. J. Kipps, T. Friedmann, Efficient infection of a human T-cell line and of human primary peripheral blood leukocytes with a pseudotyped retrovirus vector. Proc Natl Acad Sci USA, 1996. 93(21): 11842-11847.

32. Shaw, M. K., J. B. Lorens, A. Dhawan, R. Dalcanto, H. Y. Tse, A. B. Tran, C. Bonpane, S. L. Eswaran, S. Brocke, N. Sarvetnick, L. Steinman, G. P. Nolan, C. G. Fathman, Local delivery of interleukin 4 by retrovirus-transduced T lymphocytes ameliorates experimental autoimmune encephalomyelitis. Journal of Experimental Medicine, 1997. 185(9): 1711-1714.

33. Sun, L. Q., J. Pyati, J. Smythe, L. Wang, J. Macpherson, W. Gerlach, G. Symonds, Resistance to human immunodeficiency virus type 1 infection conferred by transduction of human peripheral blood lymphocytes with ribozyme, antisense or polymeric trans-activation response element constructs. Proc. Natl. Acad. Sci. USA, 1995. 92(16): 7272-7276.

34. Tough, D. F., J. Sprent, Life span of naive and memory T cells. Stem Cells (Dayt), 1995. 13(3): 242-249.

35. van den Berg, W. B., L. A. Joosten, M. Helsen, F. A. Van de Loo, Amelioration of established murine collagen-induced arthritis with anti-IL-1 treatment. Clin. Exp. Immunol., 1994. 95(2): 237-243.

36. Vikingsson, A., K. Pederson, D. Muller, Enumeration of IFN-γ producing lymphocytes by flow cytometry and correlation with quantitative measurement of IFN-γ. J. Immunol. Methods, 1994. 173(2): 219-228.

37. Walker, R., R. M. Blaese, C. S. Carter, L. Chang, H. Klein, H. C. Lane, S. F. Leitman, C. A. Mullen, M. Larson, A study of the safety and survival of the adoptive transfer of genetically marked syngeneic lymphocytes in HIV-Infected identical twins. Hum Gene Ther, 1993. 4(5): 659-680.

38. Yokota, K., N. Murata, O. Saiki, M. Shimizu, T. A. Springer, T. Kishimoto, High avidity state of leukocyte function-associated antigen-1 on rheumatoid synovial fluid T lymphocytes. J. Immunol., 1995. 155(8): 4118-4124.

What is claimed is:

1. A method of treating a human subject afflicted with a disorder characterized by the presence of a unique epitopic locus in the subject, wherein there exists a therapeutic protein which is capable of ameliorating the effects of the disorder, the method comprising the steps of
(a) isolating CD4⁺ cells from the subject;
(b) treating the isolated CD4⁺ cells so as to enrich the population of cells therein which specifically bind to the unique epitopic locus;
(c) forming transgenic CD4⁺ cells by introducing into the treated CD4⁺ cells a nucleic acid molecule encoding the therapeutic protein, wherein the nucleic acid molecule is stably transmitted to progeny CD4⁺ cells and causes the expression and extracellular placement of the therapeutic protein; and
(d) administering to the subject a therapeutically effective dose of the resulting transgenic CD4⁺ cells.

2. The method of claim 1, wherein the disorder is an inflammatory disorder.

3. The method of claim 2, wherein the inflammatory disorder is selected from the group consisting of allergic inflammation and an autoimmune disorder.

4. The method of claim 3, wherein the autoimmune disorder is selected from the group consisting of rheumatoid arthritis, insulin-dependent diabetes mellitus, multiple sclerosis, myasthenia gravis, Crohn's disease, autoimmune nephritis, primary biliary cirrhosis and psoriasis.

5. The method of claim 4, wherein the autoimmune disorder is rheumatoid arthritis.

6. The method of claim 1, wherein the therapeutic protein is selected from the group consisting of TGF_{β1}, interleukin-4, interleukin-10, interleukin-13, interleukin-1 receptor agonist, soluble interleukin-1 receptor, soluble tumor necrosis factor alpha receptor, and cow pox virus crmB.

7. The method of claim 6, wherein the therapeutic protein is IL-10.

8. The method of claim 1, wherein the nucleic acid molecule is selected from the group consisting of DNA and RNA.

9. The method of claim 1, wherein the administering is performed intravenously.

10. The method of claim 1, wherein the therapeutic protein is secreted from the cell.

11. The method of claim 1, wherein the therapeutic protein is a membrane-bound protein.

12. A pharmaceutical composition for treating a subject afflicted with a disorder characterized by the presence of a unique epitopic locus in the subject, wherein there exists a therapeutic protein capable of ameliorating the effects of the disorder, the composition comprising
(a) CD4⁺ cells derived from the subject which specifically bind to the unique epitopic locus, and which have introduced thereinto a nucleic acid molecule encoding the therapeutic protein, wherein the nucleic acid molecule is stably transmitted to progeny CD4⁺ cells and causes the expression and extracellular placement of the therapeutic protein; and
(b) a pharmaceutically acceptable carrier.

13. A method of treating a subject afflicted with a disorder characterized by the presence of a unique epitopic locus in the subject, wherein there exists a therapeutic protein which is capable of ameliorating the effects of the disorder, the method comprising the step of administering to the subject a therapeutically effective dose of the pharmaceutical composition of claim 12.

14. A kit for use in practicing the method of claim 1 comprising (a) a suitable tissue culture medium for growing CD4⁺ cells, and (b) a suitable factor for inducing CD4⁺ cell growth.
